(19) United States
(12) Patent Application Publication
(10) Pub. No.: US 2009/0286238 A1
(43) Pub. Date: Nov. 19, 2009

(54) Methods to Monitor, Diagnose and Identify Biomarkers for Psychotic Disorders

(76) Inventors: Rachel M. Craddock, Cambridge (GB); Sabine Bahn, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/095,551

(22) PCT Filed: Dec. 4, 2006
(86) PCT No.: PCT/GB2006/004509

§ 371 Date: Aug. 18, 2008

(30) Foreign Application Priority Data
Dec. 2, 2005 (GB) 0524609.5

(51) Int. Cl. C12Q 1/68; C12Q 1/02; C12Q 1/48

(52) U.S. Cl. 435/6; 435/29; 435/15

(57) ABSTRACT

A stimulated or non-stimulated T-cell sample can be used to diagnose or monitor a psychotic disorder, to identify a biomarker, or as to test a considerate as a potential therapeutic agent.

### FIELD OF THE INVENTION

The invention relates to methods for diagnosing or monitoring psychotic disorders, in particular schizophrenic disorders, using a T-cell based assay and biomarkers. The invention also relates to methods for identifying biomarkers incorporating a T-cell stimulation assay. Furthermore, the invention relates to methods for identifying agents useful in the treatment of psychotic disorders.

### BACKGROUND OF THE INVENTION

Psychosis is a symptom of severe mental illness. Although it is not exclusively linked to any particular psychological or physical state, it is particularly associated with schizophrenia, bipolar disorder (manic depression) and severe clinical depression. These conditions, their characterisation and categorisation, including DSM IV diagnosis criteria, are described in PCT/GB2006/003870, the content of which is incorporated herein by reference.

WO01/63295 describes methods and compositions for screening, diagnosis and determining prognosis of neuropsychiatric or neurological conditions (including bipolar affective disorder, schizophrenia and vascular dementia), for monitoring the effectiveness of treatment in these conditions and for use in drug development.

Other techniques such as magnetic resonance imaging or positron emission tomography based on subtle changes of the frontal and temporal lobes and the basal ganglia are of little value for the diagnosis, treatment, or prognosis of schizophrenic disorders in individual patients, since the absolute size of these reported differences between individuals with schizophrenia and normal comparison subjects has been generally small, with notable overlap between the two groups. The role of these neuroimaging techniques is restricted largely to the exclusion of other conditions which may be accompanied by schizophrenic symptoms, such as brain tumours or haemorrhages.

The validation of biomarkers that can detect early changes specifically correlated to reversal or progression of mental disorders is essential for monitoring and optimising interventions. Used as predictors, these biomarkers can help to identify high-risk individuals and disease sub-groups that may serve as target populations for chemo-intervention trials; as surrogate endpoints, biomarkers have the potential for assessing the efficacy and cost effectiveness of preventative interventions at a speed which is not possible at present when the incidence of manifest mental disorder is used as the endpoint.

WO2005/020784 discloses surrogate cell gene expression signatures, by a minimally invasive technique for determining the prognosis of a subject or the subjects susceptibility to a disease, disorder or physical state. It is reported (in Example 2) that various genes are modulated in, inter alia, psychiatric illness.

Therefore, a need exists to identify sensitive and specific methods and biomarkers for diagnosis and for monitoring psychotic disorders, such as schizophrenic or bipolar disorders. Additionally, there is a clear need for methods, models, tests and tools for identification and assessment of existing and new therapeutic agents for the treatment of these disorders and methods for diagnosing psychotic disease.

T-cells are lymphocytes which develop in the thymus and play an important role in the immune system. There are two sub-populations of T-cells: cells with a CD4 marker are called helper T-cells whilst CD8+ cells are cytotoxic T-cells. Both T-cell types have a T-cell receptor (TCR) for antigen recognition. Stimulation or activation of a resting T-cell is initiated by the interaction of the TCR-CD3 complex with antigen-MHC class II molecules on the surface of an antigen-presenting cell. This interaction initiates a cascade of biochemical events in the T-cell, including activation of gene transcription, that eventually results in growth, proliferation and differentiation of the T-cell.

### SUMMARY OF THE INVENTION

This invention is based at least in part on the discovery that assays, conducted on stimulated or unstimulated T-cells, can provide valuable information on the condition of a subject. T-cells provide a good model in which to investigate cellular function, as they are relatively easy to isolate, e.g. from peripheral blood, with high purity and can be obtained in a minimally invasive fashion.

One aspect of the present invention is a method of diagnosing or monitoring a psychotic disorder in a subject, comprising:

a. providing a test T-cell sample from the subject;

b. providing a stimulus to the test T-cell sample; and

c. assessing a response to the stimulation.

This method can be utilised in assessing prognosis of a psychotic disorder. It can also be used in a method of monitoring efficacy of a therapeutic substance in a subject having, suspected of having, or not being predisposed to, a psychotic disorder.

A second aspect of the present invention is a method of identifying a biomarker of a psychotic disorder, comprising:
a. providing a test T-cell sample from a subject having a psychotic disorder;
b. providing a stimulus to said test T-cell sample;
c. assessing a response to the stimulus;
d. comparing the response with a response to stimulus in a control T-cell sample; and
e. detecting any difference in the responses, thereby identifying a biomarker.

A third aspect of the present invention is a method of testing for a potential agent for therapy of a psychotic disorder, which comprises:

a. providing a test T-cell sample from a subject having a psychotic disorder;

b. contacting the test T-cell sample with a candidate agent;

c. providing a stimulus to the test T-cell sample; and

d. assessing a response to the stimulation.

A fourth aspect of the present invention is a method of diagnosing or monitoring a psychotic disorder in a subject, comprising:
a. providing a test T-cell sample from the subject; and
b. comparing gene and/or protein expressions in the test sample with a control sample.

A further aspect of the invention is a sensor, e.g. a biosensor, as defined below. In a method of the invention, biomarkers can be detected using a sensor comprising one or more enzymes, binding receptor or transporter proteins, antibodies, antibody fragments, synthetic receptors or other selective binding partners such as aptamers and peptides for the direct or indirect detection of biomarkers. The recognition element of the sensor may be coupled to an electrical, optical, acoustic, magnetic or thermal transducer or to a microengineered system associated with the transducer or to a nanoparticulate system such as quantum dots or surface plasmon particles.

### DESCRIPTION OF PREFERRED EMBODIMENTS

For the avoidance of doubt, terms such as “response”, “control” and “sample” as used herein include the possibility of there being more than one such response, control or sample, respectively.

The term “diagnosis” as used herein encompasses identification, confirmation, and/or characterisation of a psychotic disorder, in particular a schizophrenic disorder, bipolar disorder, related psychotic disorder, or predisposition thereto. By predisposition it is meant that a subject does not currently present with the disorder, but is liable to be affected by the disorder in time.

Monitoring methods of the invention can be used to monitor onset, progression, stabilisation, amelioration and/or remission of a psychotic disorder.

The term “psychotic disorder” as used herein refers to a disorder in which psychosis is a recognised symptom, this includes neuropsychiatric (psychotic depression and other psychotic episodes) and neurodevelopmental disorders (especially autistic spectrum disorders), neurodegenerative disorders, depression, mania, and in particular, schizophrenic disorders (paranoid, catatonic, disorganized, undifferentiated and residual schizophrenia) and bipolar disorders. Preferably, the invention relates to schizophrenic disorders.

T-cell samples are preferably obtained from peripheral blood taken from a subject. Preferably, T-cell samples are freshly isolated, that is they are used immediately following sample collection.

An example of a method for T-cell isolation is described herein (Example 1). However, the skilled person will appreciate that other methods known in the art for obtaining or isolating T-cells from a biological sample, such as peripheral blood, may also be employed.

The term “stimulus” as used herein refers to a stimulus capable of inducing a response, preferably T-cell proliferation and responses associated with T-cell receptor-triggering.

In vitro T-cell stimulation may be used as a method of comparing the functional responses of patient and control cells, firstly in order to investigate peripheral evidence of disease processes in schizophrenia and also to investigate whether global abnormalities or deficits in cell processes, such as cell signalling, gene transcription, protein synthesis and trafficking underlie the pathophysiology of this disorder. in vivo T-cell activation involves ligation of the T-cell receptor (TCR) through interaction with specific antigen presented in association with MHC. The TCR signalling complex is composed of a number of molecules including CD3, which provides the cytoplasmic signalling function of the complex, CD45, involved in de-phosphorylation of inhibitory phosphorylated tyrosine motifs and either CD4 or CD8, which are believed to stabilise the signalling complex. For optimal T-cell responses, co-stimulation is preferred for amplification and regulation of the initial signal. This is provided by molecules such as CD28, CD40, CD80/CD86 and OX40L.

Preferably, stimulation of T-cells is carried out in vitro by mimicking a TCR signal via cross-linking of cell surface CD3, using a monoclonal antibody (anti-CD3). This ultimately results in cell cycle entry and, as T-cell stimulation induces transcription factor activation, gene transcription, protein synthesis and protein trafficking, methods of the invention aim to identify and trace any abnormalities in these physiological processes and any consequences (e.g. differences in response to stimulus which may manifest in differences in mRNA, protein, lipid or other metabolite levels or ratios associated with such abnormalities).

Preferably, the stimulus is anti-CD3 antibody. Stimulation of T-cells may also be carried out using other agents, for example ionomycin and PMA, alone or in combination with CD3.

The term “response” as used herein may thus refer to a response elicited in response to the stimulation/activation of a resting T-cell. Such responses include proliferation, transcription factor activation or deactivation and modulation of one or more of the following: gene expression, protein synthesis, signal transduction, cytokine synthesis, protein trafficking and protein turnover, metabolite or lipid profile. Preferably, the response comprises proliferation, modulation of gene expression, protein synthesis and/or protein turnover.

Identification of differences between responses in T-cell samples from a subject having or being predisposed to a psychotic disorder, and stimulatory response in a normal subject, not affected by or predisposed to a psychotic disorder, can therefore be used to diagnose or monitor psychotic disease. Methods of the invention may comprise comparing a response in a test T-cell sample from a subject with a response to stimulation in a control. Suitable controls include normal controls derived from individuals not unaffected by or predisposed to psychotic disorder and disorder controls derived from individuals with a psychotic disorder preferably a schizophrenic disorder.

Methods of the invention may comprise detecting a difference in a response between the test sample and a control sample.

Thus, methods of the invention may involve comparing a response in a test T-cell sample with a response in a normal control T-cell sample, wherein a difference in response is indicative of the presence of or predisposition to a psychotic disorder such as a schizophrenic disorder. Differences in response may be detected as a presence, absence, increase or decrease in a particular response to stimulus.

Alternatively or additionally, methods of the invention may comprise a response in a test T-cell sample with a response in a psychotic disorder control T-cell sample, to enable the test T-cell response to be matched to the response characteristic of a particular psychotic disorder; such comparisons are useful for differential diagnosis of psychotic disorders that present with similar or overlapping clinical symptoms.

Following stimulation, T-cells from schizophrenia patients have been found to have significantly lower proliferation compared to healthy controls, as illustrated in Example 2. Thus, in those embodiments where the response is proliferation, a lower proliferation in a T-cell sample from a subject compared to proliferation in a normal control T-cell sample is indicative of a psychotic disorder, in particular schizophrenia, being present. Proliferation may be assessed by ³[H]-thymidine incorporation into progeny cell DNA, as illustrated in Example 1.

Differences in responses of T-cells from individuals having or predisposed to psychotic disorders and those from normal individuals may also be detected by assessing modulation in gene expression in response to exposure to stimulus, preferably in response to exposure to a stimulus for T-cell proliferation. Differences in responses may also be assessed by considering the downstream effects of differential gene expression in subjects having or being predisposed to a psychotic disorder, e.g. differences in metabolic profile, lipid profile, or differences in levels or ratio of biomarkers, compared to those in normal individuals not suffering from or predisposed to a psychotic disorder.

The terms “modulated” and “modulation” are used herein to mean an upregulation or downregulation of expression of a gene or differences in the proteome, for example, an increase or decrease in protein level. Modulation of gene expression can be measured by detecting a variation in mRNA or protein levels. The increase or decrease in protein level may be assessed by simply determining the presence or absence of a protein or by using a quantitative method.

Methods of determining the expression level of a gene are well known in the art. According to the methods of the invention, modulation of expression can be identified by assessing the amount or concentration of mRNA, a nucleic acid derived from mRNA or a protein translated from the mRNA. Gene expression may be measured by assessing mRNA levels using a method including reverse transcription and polymerase chain reactions (“RT-PCR”), such as quantitative PCR (in particular, real-time quantitative PCR), and Northern blotting. In one suitable method for determining the level of mRNA expressed, a total RNA sample is obtained from the cell, cDNA is synthesized from mRNA of the gene or genes of interest, and the cDNA is used for real-time quantitative PCR analysis to determine the level of the mRNA of interest in the sample. Systems and kits for implementing such methods are commercially available.

Arrays may be used to assess expression of a plurality of genes or proteins, for example using weak cation exchange (CM10) chips for SELDI analysis of proteins, or Codelink Bioarrays for gene expression. An example of a method used to assess gene expression is shown in Example 3.

Examples of suitable methods for determining the level of protein expression or identifying protein biomarkers include immunological methods, involving an antibody, or an antibody fragment capable of specific binding to the protein of interest. Suitable immunological methods include sandwich immunoassays, such as sandwich ELISA in which detection of the peptide is performed using two antibodies which recognize different epitopes; radioimmunoassays (RIA), direct or competitive enzyme-linked immunosorbent assays (ELISA), enzyme-immuno assays (EIA), Western blotting, immunoprecipitation and any particle-based immunoassay (e.g. using gold, silver, latex or magnetic particles or Q-dots). Immunological methods may be performed, for example, in microtitre plate or strip format.

Other techniques that may be used in the methods of the invention, for example for the detection, identification and/or quantification of a biomarker, e.g. for quantifying the level of a nucleic acid, protein, lipid or metabolite present, include spectral analysis, such as NMR spectroscopy and high resolution NMR spectroscopy (¹H NMR), mass spectrometry, such as Surface Enhanced Laser/Desorption Ionization (SELDI) (-TOF) and/or MALDI (-TOF), 1-D gel-based analysis, 2-D gel-based analysis, LC-MS-based technique or iTRAQ™. An example used to analyse proteins is shown in Example 4.

iTRAQ™ technology involves the chemical tagging of N-terminus peptides resulting from protein digestion with trypsin. Up to four labelled samples are combined, fractionated by nano-LC and analysed by tandem mass spectrometry. Protein identification is then achieved by database searching of fragmentation data. Relative quantification of peptides is achieved by fragmentation of the chemical tag, which results in a low molecular weight reporter ion. As samples are labelled after tryptic digestion, analysis of high molecular weight proteins such as trans-membrane receptors is possible and quantification of fragmented tag provides greater confidence in protein identity and quantification.

According to the invention, a suitable cohort of patients and controls may be selected including first onset and/or minimally treated individuals and these will be compared with chronically ill patients having a more established clinical history. This allows comparison of both disease progression and the effects of drug treatment. Membrane-bound and soluble proteins may be prepared from stimulated T-cells. Thus, proteomic profiling of T-cells from psychosis patients and controls may be performed, providing information regarding differing expression of large and small molecular weight and proteins, e.g. phosphoproteins, following stimulation.

Methods of the invention may comprise comparing samples by assessing variation in one or more biomarkers in response to stimulation of the sample. The term “biomarker” means a distinctive biological or biologically-derived indicator of a process, event, or condition. Biomarkers can be used in methods of diagnosis (e.g. clinical screening), prognosis assessment; in monitoring the results of therapy, identifying patients most likely to respond to a particular therapeutic treatment, drug screening and development. Preferably, the biomarker is a gene, mRNA, a protein or peptide, lipid, or metabolite. The terms protein and peptide are used interchangeably herein. The biomarker may be quantified. Biomarkers and uses thereof are valuable for identification of new drug treatments and for discovery of new targets for drug treatment. Quantifying the amount of the biomarker present in a sample may include determining the concentration of the peptide biomarker present in the sample. Detecting and/or quantifying may be performed directly on the sample, or indirectly on an extract therefrom, or on a dilution thereof. Detecting and/or quantifying can be performed by any method suitable to identify the presence and/or amount of a specific protein in a biological sample.

In one embodiment, the control sample comprises a normal control sample. In another embodiment, the control sample comprises a psychotic disorder control sample. In another embodiment, the method may also comprises classifying proliferative responses of a sample as having a normal profile, psychotic disorder profile, or psychotic disorder predisposition profile.

In methods of the invention, in particular those for diagnosing and monitoring, T-cell samples may be taken on two or more occasions from a test subject. Stimulatory responses from samples taken on two or more occasions from a test subject can be compared to identify differences between the stimulatory responses in samples taken on different occasions. Methods may include analysis of stimulatory responses from biological samples taken on two or more occasions from a test subject to quantify the level of one or more biomarkers present in the biological samples, and comparing the level of the one or more biomarkers present in samples taken on two or more occasions.

Diagnostic and monitoring methods of the invention are useful in methods of assessing prognosis of a psychotic disorder, in methods of monitoring efficacy of an administered therapeutic substance in a subject having, suspected of having, or of being predisposed to, a psychotic disorder and in methods of identifying an anti-psychotic or pro-psychotic substance. Such methods may comprise comparing the level of the one or more biomarkers in a test biological sample taken from a test subject with the level present in one or more samples taken from the test subject prior to administration of the substance, and/or one or more samples taken from the test subject at an earlier stage during treatment with the substance. Additionally, these methods may comprise detecting a change in the level of the one or more biomarkers in biological samples taken from a test subject on two or more occasions.

A method of diagnosis of or monitoring according to the invention may comprise quantifying the one or more biomarkers in a test biological sample taken from a test subject and comparing the level of the one or more biomarkers present in said test sample with one or more controls. The control can be selected from a normal control and/or a psychotic disorder control. The control used in a method of the invention can be selected from: the level of biomarker found in a normal control sample from a normal subject, a normal biomarker level; a normal biomarker range, the level in a sample from a subject with a schizophrenic disorder, bipolar disorder, related psychotic disorder, or a diagnosed predisposition thereto; a schizophrenic disorder marker level, a bipolar disorder marker level, a related psychotic disorder marker level, a schizophrenic disorder marker range, a bipolar disorder marker range and a related psychotic disorder marker range.

Detecting differences in responses enables identification of biomarkers for a psychotic disorder. The response may be assessed by any suitable method or combination of methods, for example by considering gene expression, at the mRNA and/or protein level, to detect differential gene expression between disorder and control samples, by considering protein levels (e.g. in cell lysate), lipid profile and/or metabolite profile. The differences may manifest as the presence or absence of a biomarker or a difference (increase or decrease) in level of a biomarker, or in ratios of a biomarker or biomarkers.

Differences in gene expression can be detected by a modulation in mRNA or protein levels. Where the biomarker is a gene, the expression of the gene present in the disorder sample may be modulated compared to the expression of the gene in the control sample, thus different levels of mRNA transcribed from the gene will be detected. For example, the expression may be increased or decreased, or different splice variants or ratios of splice variants of the mRNA may be detected. In another embodiment, the biomarker is a protein and the level of the protein present in the sample differs from the level of the protein present in the control sample. For example, the level may be modulated so that it is increased or decreased, or a difference in protein cleavage products may be found; this may be assessed by a quantitative method or determined by the presence or absence of the protein.

In one embodiment, the level or ratio of one or more biomarkers is detected. This may be carried out using a sensor, e.g. a biosensor comprising one or more enzymes, binding, receptor or transporter proteins, antibody, synthetic receptors or other selective binding molecules for direct or indirect detection of the biomarkers. For detection, the sensor may be coupled to an electrical, optical, acoustic, magnetic or thermal transducer.

The term “antibody” as used in this embodiment includes, but is not limited to, polyclonal, monoclonal, bispecific, humanised or chimeric antibodies, single chain antibodies, Fab fragments and F (ab′)₂ fragments, fragments produced by a Fab expression library, anti-idiotypic (anti-id) antibodies, and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

Biomarkers identified using a method of the invention can be used as biomarker for a psychotic disorder or predisposition thereto. They are thus useful in methods for monitoring or diagnosing psychotic disease.

The present invention may be used identify a potential therapeutic agent for the prevention, treatment or amelioration of a psychotic disorder. In one embodiment, the invention comprises comparing a response to stimulation with a response in a control sample. In particular, responses in test and normal control T-cell samples exposed to a candidate therapeutic agent may be compared, identifying the candidate as a potential therapeutic agent if one or more responses in the test T-cell sample are modulated such that a normal response is restored.

According to this aspect, a candidate therapeutic agent is identified if the candidate therapeutic agent is capable of modulating a response in T-cells from a subject having a psychotic disorder, in particular such that one or more responses are restored to the response characteristic of T-cells from normal individuals. Preferably, the response is proliferation or modulation of gene expression, i.e. changes in mRNA or protein levels. The response can be assessed using the methods described herein, in particular by assessing biomarkers of response identified as described herein. Changes in proliferation can be assessed by comparing the proliferation of T-cells in the presence and absence of the candidate therapeutic agent. Modulation of expression of one or more genes can be assessed by comparing the expression level of the gene or genes (at the mRNA or protein level) in the presence and absence of the candidate therapeutic agent. Modulation of protein levels can be assessed by comparing the level of the protein or proteins in the presence and absence of the candidate therapeutic agent. Other suitable biomarkers of response include lipids and metabolites found at different levels in disorder and normal control samples.

As shown in Example 5, a proliferative response can be restored using co-stimulation with CD28. Accordingly, co-stimulation with CD28 may be used as a control for restoring proliferative responses. Co-stimulation with CD28 is also a useful approach for dissecting the pathophysiology of a psychotic disorder.

The above discussion focuses on responses to stimulation of T-cells. In the fourth aspect of the invention, stimulation is not necessary. By comparison, with a control, a psychotic disorder can be diagnosed or monitored, e.g. by identifying modulation of gene expression and/or the presence or absence of one or more proteins. Illustrative procedures for this purpose are described above.

One altered transcript that has been found, pertaining to cell cycle, is STAT 1, which is increased in schizophrenia patients. STAT 1 is involved in cytokine signalling and interacts with the transcription factor NFκB. An increase in RBL2 and a decrease in RBL1 expression are found in patient samples, where proliferative responses were significantly lower. The function of these gene products is governed by phosphorylation by GSK3B, which is also found to be up-regulated.

There is altered expression of the dystonin and dystrobrevin genes in schizophrenia patients. Dystonin acts as a cytoskeletal linker protein, interacting with actin and microtubules and functioning to stabilise chromosomes.

CDCL5 and NLK are also found to be altered in schizophrenia. Although annotated in signal transduction categories, they are also involved in cell cycle regulation.

Functional pathways involved in signal transduction are significantly altered. There is differential expression of glutathione peroxidase 7, thioredoxin 2 and ferredoxin reductase, altered expression in schizophrenia of cytochrome c oxidase subunit Va (up-regulated in schizophrenia) and cytochrome b-5 (down-regulated) and particularly ACADVL (up-regulated) and ACAD9 (down-regulated). These are acyl-CoA dehydrogenase enzymes involved in β-oxidation of fatty acids, used as an alternative energy source in the consequence of lower glucose availability. Ankyrin 1 and ankyrin 2, cytoskeletal elements more commonly associated with red cell spherocytosis are also both down-regulated. GADD45A, found to be up-regulated in schizophrenia, interacts with elongation factor 1a and actually disrupts cytoskeletal stability.

In another aspect, the invention relates to a diagnostic kit or monitoring kit suitable for performing a method described herein. Kits according to the invention may comprise one or more components selected from: instructions for use of the kit, one or more normal and/or psychotic disorder controls, a sensor or biosensor suitable and/or adapted for detecting a biomarker according to the invention and a ligand, e.g. nucleic acid, antibody, aptamer, or the like, capable of specifically binding a biomarker according to the invention or specifically binding a substance derived from the biomarker or from the action of the biomarker. The ligand may be provided immobilised on a solid support such as bead or surface, for example in the form of an array adapted for use in a method of the invention.

The following Examples illustrate the invention.

### Example 1

### Isolation of T-Cells

All experiments involved were carried out on CD3+ T-cells (including CD4+ and CD8+ cells, all heterogeneous with regard to activation state). T-cells were isolated from the peripheral blood of schizophrenia patients and age, sex and race-matched controls.

Peripheral blood mononuclear cells (PBMCs) were isolated by centrifugation of peripheral blood over Ficoll-paque (Amersham) in 50 ml tubes at 750×g for 20 minutes. PBMCs were removed from the plasma/Ficoll interface using a sterile Pasteur pipette and transferred into a clean 50 ml tube containing PBS. These cells were washed three times in PBS and counted using a haemocytometer. T-cells were purified from PBMCs using MACS human T-cell isolation kit (Miltenyi Biotech) by following the manufacturer's protocol. CD3+ T-cells were then washed twice in RPMI medium (Sigma), counted and cultured at 2.5×10⁶ cells/ml in complete T-cell medium (RPMI, 10% foetal calf serum, 1% penicillin/streptomycin/glutamine).

### Example 2

### In Vitro Stimulation of T-Cells

in vitro T-cell stimulation was carried out using anti-CD3 (clone OKT3) alone. This is the simplest method of in vitro stimulation, as this antibody serves to bring together all the components of the TCR to effect a signal. Subsequent experiments to further explore T-cell responses with co-stimulation were carried out using anti-CD3+anti-CD28, anti-CD3+IL-2, antiCD3+PBMCs, and with PMA and ionomycin.

Stimulation of T-cells in vitro with anti-CD3 was carried out in 96-well round-bottom tissue culture plates (Nunc), coated with 0, 0.01, 0.1 and 1 μg/ml OKT3 in PBS for 1 hour at 37° C. Plates were washed with PBS before the addition of 0.2×10⁶ T-cells in 200 μl complete T-cell medium.

Proliferative responses to stimulation were measured using ³[H]-thymidine incorporation into progeny cell DNA. In brief, T-cells were cultured for 48 hours at 37° C. in CO₂ incubator and pulsed with 1 μCi ³[H]-thymidine per well for 24 hours. Cells were harvested onto 96 well filter plates to capture labelled DNA and fluorescent scintillation fluid was used to measure ³[H]-thymidine incorporation.

T-cells were obtained from schizophrenia patients. They included chronic, clozapine-treated patients, i.e. patients who have been receiving clozapine therapy for a number of years; minimally-treated patients, i.e. patients receiving treatment other than clozapine for less than 2 months, or non-compliant with treatment; and untreated, recently diagnosed schizophrenics. All were found to have significantly lower proliferative responses compared to controls at all concentrations of anti-CD3. This not only provided evidence that peripheral differences between schizophrenia patients and controls can be observed in peripheral tissues, but also supplied a model for dynamic investigations into the role of cellular dysfunction in this disorder.

### Example 3

### Codelink Gene Array Analysis

Patient and control samples were made from 3×10⁶ freshly isolated T-cells, and from cells cultured for 24 hours in the presence of 1 μg/ml anti-CD3.

Total RNA was extracted from these samples using QIAamp RNA blood mini kit (Qiagen). RNA quality was checked using Agilent lab-on-a-chip nanochips and quantified using a Nanodrop system.

RNA was prepared for hybridisation to Codelink gene array chips using the Codelink Expression Bioarray System according to the manufacturer's instructions and using the recommended reagents. In brief, 1 μg total RNA was used for first strand synthesis and, following second strand synthesis, double stranded cDNA was purified using QIAquick PCR purification kit (Qiagen). Biotin-labelled cRNA was synthesised using the in vitro transcription reagents provided within the kit and subsequently purified using RNeasy mini kit (Qiagen). The cRNA concentration was measured using the Nanodrop system and quality was checked using Agilent lab-on-a-chip. 10 μg biotin-labelled cRNA was hybridized to Codelink array slides by following the manufacturer's protocol. Slides were washed and scanned using a GenePix Personal 4100A Microarray Scanner.

### Example 4

### Differential Protein Expression

Cells were cultured for 48 hours at a density of 2.5×10⁶ cells/ml in 24 well tissue culture plates (Nunc). Cells were then transferred to 1.5 ml microfuge tubes and washed once in PBS before storage at −80° C. 5×10⁶ cells/sample were lysed in 250 μl binding buffer (9 M urea, 50 mM hepes, 2% chaps, pH7) containing protease and phosphatase inhibitors (Roche complete inhibitor cocktail, orthovanadate, pyrophosphate, glycerophosphate, NaF). Samples were vortexed for 5 seconds and left to lyse on ice for 10 minutes. Samples were vortexed again and centrifuged at 13,000 rpm for 5 minutes at 4° C. to remove cell debris. A 40 μl aliquot of each sample was loaded onto Ciphergen CM10 weak cation exchange chips, following the manufacturer's protocol, using pH7 binding buffer described above. Chips were air-dried, and 1.2 ml sinopinic acid (SPA) was added to each spot before analysis in the ProteinChip Reader.

In order to identify peaks that were different between groups, a feature extraction and classification process was performed. The feature extraction consisted of four steps: peak identification, alignment, windowing and quantification. Peak Identification involves deciding the points at which the derivative changes sign. Peak alignment requires identification of the largest peak which is present in all spectra. The spectra were all shifted such that this peak occurred at the same point in all spectra. This accounts for global shifts due to calibration drifts.

Windowing was performed by centering a window around each peak. Overlap error was accounted for by considering groups of windows which overlap, then identifying the largest peak within this region, centering a window around this peak and then placing further windows in both directions until the original region was covered. Finally, a trapezoidal integration over each window was performed and the set of such values was considered the feature set. Classification may be performed by any number of standard techniques, such as linear discriminant analysis, discriminant trees, boosting, support vector machines or artificial neural networks. In this case partial least squares discriminant analysis (PLS-DA) was performed to identify those peaks which were significantly different between sets. PLS-DA analysis allowed for complete separation of patient and control groups.

Several differentially expressed peaks between patient and control groups and between patient and control responses were identified using this method. Protein identification was performed using two methods. For peaks of 4 KDa and smaller, direct sequencing was carried out using SELDI MS MS. Proteins larger than 4 KDa were identified by gel electrophoresis and LC MS MS sequencing. Initially hydrophobic or cationic protein fractionation was carried out on the cell lysates to simplify the protein constituents.

50 μl PLRP-S reversed phase beads (Polymer laboratories) were equilibrated with 10% ACN/0.1% TFA, spun down and the supernatant removed, whilst T-cell lysates, prepared as described above, were adjusted to contain a concentration of 10% ACN, 0.1% TFA in a volume of 400 μl and added to PLRP-S beads. Proteins were allowed to bind to beads for 30 minutes at room temperature on a rotator. Samples were then spun for 1 minute at 5000 rpm to remove supernatant, and were then washed 3 times in 10% ACN, 0.1% TFA. Supernatant was removed and successive protein fractions were eluted with 400 μl 20%, 30%, 40%, 50%, 60%, 70%, 80%, 90% and 100% ACN in 0.1% TFA. Each fraction Was collected into 1.5 ml microfuge tubes and 2 μl was profiled for desired peaks using NP20 chips in accordance with the manufacturer's protocol.

Fractions were chosen according to expression of the desired peaks and these were pooled and concentrated in a Speed vac for 2 hours at 30° C., until completely dry. Fractions were run on non-denaturing gels; the band of desired molecular weight was cut out, trypsinised overnight and submitted for LC MS MS sequencing.

Cationic protein fractionations were prepared by the application of T-cell lysates (as described earlier) to CM10 columns (Ciphergen) for 2 hours at 4° C. This allowed specific binding of the proteins originally profiled using CM10 chips (Ciphergen). Columns were washed twice with lysis buffer to remove unbound proteins, whilst proteins of interest were eluted using lysis buffer at pH 11. Buffer exchange to 50 mM Tris pH7 was carried out, using 5 KDa molecular weight cut-off spin columns. Eluates were concentrated in a Speed vac for 2 hours at 30° C., until completely dry, and proteins of interest were separated according to molecular weight on non-denaturing SDS-PAGE gels. The band of desired molecular weight was cut out, trypsinised overnight and submitted for LC MS MS sequencing.

### Example 5

### Co-Stimulation

T-cells were also co-stimulated by the addition of 10 ng/ml IL-2, anti-CD28 and PBMCs, in order to determine if proliferative responses could be restored using more complex methods of stimulation. These were added directly to T-cells cultured in anti-CD3 coated plates, as described above. Anti-CD3 stimulation of T-cells in the presence of all co-stimulatory molecules present on B cell and monocyte antigen presenting cells was tested by the incubation of PBMCs, isolated by centrifugation over Ficoll-paque as described above, in wells coated with anti-CD3 at 0.26×10⁶ cell/ml, allowing for 70% CD3+ T-cells in PBMCs. Downstream pathways of T-cell activation were also tested by stimulation in culture with 50 ng/ml phorbol myristate acetate (PMA) and 500 ng/ml ionomycin, which directly activate PKC and calcium fluxes respectively. This was also carried out in a volume of 200 μl in 96 well plates. Following in vitro co-stimulation, T-cell proliferation was measured as described above.

Patient T-cell responses to co-stimulation with anti-CD3 and anti-CD28 were not found to be significantly different compared to healthy control responses, suggesting that co-stimulation through CD28 can restore patient responses.

### Example 6

### Sample Collection

Peripheral blood was taken from clozapine-treated chronically ill patients, who met DSM-IV criteria for a diagnosis of schizophrenia and minimally treated patients with a determined diagnosis of schizophrenia who had either had less than 4 weeks of therapy, or who were non-compliant with drug therapy. Blood was also taken from drug-naïve patients with first onset psychosis, who presented with clinical symptoms consistent with an eventual diagnosis of schizophrenia. Blood was taken from age, sex and race-matched controls for each patient and processed concomitantly. The demographic details are shown in Table 1.

TABLE 1

| | | Proliferation | | Minimally treated | | Microarrays | |
|---|---|---|---|---|---|---|---|
| | | Control | Patient | Control | Patient | Control | Patient |
| Age | | 34 ± 10.5 | 35 ± 10.8 | 29 ± 6.9 | 28 ± 10.4 | 30 ± 6.4 | 31 ± 14.1 |
| Sex | Male | 21 | 35 | 10 | 9 | 3 | 2 |
| | Female | 11 | 4 | 2 | 2 | 3 | 4 |
| Race | White | 24 | 35 | 7 | 7 | 6 | 6 |
| | Black | 1 | 3 | 1 | 1 | 0 | 0 |
| | Asian | 4 | 1 | 4 | 3 | 0 | 0 |
| | Oriental | 1 | 0 | 0 | 0 | 0 | 0 |
| Smoking | Smoker | 12 | 20 | 2 | 4 | 2 | 4 |
| | Non-smoker | 15 | 7 | 10 | 5 | 4 | 2 |
| | Not known | 5 | 12 | 0 | 2 | 0 | 0 |

### T-Cell Isolation

CD3+ T-cells were isolated from the peripheral blood of schizophrenia patients and age, sex and race-matched controls. In brief, peripheral blood was taken using S-monovette blood collection system containing EDTA (Sarstedt). Mononuclear cells (PBMC) were isolated by centrifugation over Ficoll-Paque (Amersham Biosciences, Amersham, UK) and CD3+ pan T-cells were then purified from these by negative selection using MACS human pan T-cell isolation kit in association with LS separation columns (Miltenyi Biotech, UK). T-cell purity was routinely above 99%, when analysed for CD3-ε expression by flow cytometry (FACS Calibur, Becton Dickinson). Where indicated, cells were cultured at 37° C. in RPMI medium containing 10% foetal bovine serum and 1% penicillin, streptomycin and glutamine (Sigma, UK).

### T-Cell Proliferation

Proliferative responses to stimulation were measured using ³H-thymidine incorporation into progeny cell DNA. T-cells were cultured for 48 hours in 96 well plates coated with 0, 0.01, 0.1 and 1 μg/ml anti-CD3 (clone OKT3), seeded at a density of 2×10⁵ cells/well, to stimulate entry into the cell cycle. Cells were pulsed with of 0.037 MBq (1 μCi) ³H-thymidine (Amersham Biosciences, UK) per well for a further 24 hours to allow incorporation into DNA and harvested onto 96 well filter plates (Perkin Elmer) to capture labelled DNA. Labelled DNA and hence proliferation was measured using a scintillation counter (Top Count, Packard). Statistical significance was determined using a non-parametric Mann-Whitney U test, with a P-value of less than 0.05 considered significant.

### Analysis of CD3 Expression on Patient and Control T-Cells

Patient and control T-cells were cultured for 72 hours in the presence or absence of 1 μg/ml plate-bound anti-CD3. Cells were counted and 5×10⁵ cells/sample were washed 3 times in FACS buffer (PBS, 2% foetal bovine serum, Sigma, UK) and resuspended in 100 μl FACS buffer containing anti-CD3 conjugated to Cy5. Cells were incubated at 4° C. for 20 minutes before further washing in FACS buffer. Cells were counted using FACS Caliber and Cell Quest software (BD Bioscinces, UK). Data were analysed using WinMDI (Purdue University, Indiana) and Flowio (Treestar). Statistical significance was determined using the Mann-Whitney U Test.

### Microarray Analysis of T-Cell Gene Expression

Differential gene expression between T-cells from six minimally treated schizophrenia patients and six age, sex and race matched controls was investigated using CodeLink™ Human Whole Genome Bioarrays (GE Healthcare, UK). Total RNA was extracted from freshly isolated T-cells using QIAamp RNA blood mini kit (Qiagen, UK) and quality was assessed with a high-resolution electropheresis system (Agilent Technologies, Palo Alto, Calif., USA). Biotin-labelled cRNA was generated from each sample following the manufacturer's protocol. cRNA was hybridised onto CodeLink whole genome microarray slides, washed and hybridised cRNA species were detected using Cy5-Streptavidin (Ameraham, UK). Slides were scanned using GenePix Personal 4100A Microarray Scanner (Axon Instruments) and analysed with CodeLink Expression Analysis software.

### Preprocessing and Normalization of Microarray Data

Probe sets were initially filtered to include only those with signal above background noise level, using the strict criterion that probes must be flagged ‘good’ by the Codelink software on all chips in the experiment. This reduced the number of probes included in the analysis from 54000 to 12416. The spot mean signal intensities for these probes were read into the R statistical program (http://www.r-project.org/) for further analysis, using Bioconductor (1) packages where appropriate. Data were normalised using the ‘quantile’ method (2) and quality control (QC) procedures were performed to identify outlier chips. These included analysis of pairwise correlations of normalised expression values for all chips, boxplots of the normalised expression values for all chips and comparing each chip to a pseudo-median chip. One outlier was identified and removed from further analysis. The remaining samples were then re-normalised.

### Detection of Differentially Expressed Genes in Freshly-Isolated T-Cells

Paired t-tests were carried out between patients and controls, using the Limma package (linear models for microarray data) to identify differential expression (2, 3). A correction for multiple testing was applied with the ‘qvalue’ package (4) and probes with q<0.05 were considered differentially expressed.

### Functional Profiling of Significantly Altered Transcripts

Pathway analysis to characterise the genes significantly altered in freshly-isolated T-cells from schizophrenia patients compared to controls was carried out using Onto-Express. Significant probes sets were first mapped to corresponding Entrez IDs using Onto-Translate (5) and this output submitted to Onto-Express. Default settings were used with the Codelink human whole genome as the reference array. Biological process categories with a corrected p<0.05 and containing more than 2 genes were selected as the most important pathways affected in the disease state.

### Chromosomal Mapping

Chromosomal mapping and analysis was performed using an in-house algorithm, initially designed for use with Affymetrix data. Codelink probe IDs were therefore first mapped to Affymetrix probe IDs using Onto-Translate (5). The main steps in the analysis were: (i) selection of a representative probe-set for each gene, which was mapped to its alignment region and discarded if it did not correspond to the location of the target gene; (ii) assessment of distribution of differentially expressed genes across the genome using a sliding window; (iii) assignment of a score to each window, based on the binomial distribution such that high scores corresponded to regions containing an excess of differentially expressed genes; (iv) identification of chromosomal regions with a high proportion of differentially expressed genes, which may be of biological significance.

Proliferative Responses to Stimulation with Anti-CD3 are Significantly Lower in Patients with Schizophrenia.

³H-thymidine incorporation was used to measure the proliferation of peripheral blood T-cells from 39 patients and 32 controls, treated with 0, 0.01, 0.1 and 1 μg/ml anti-CD3. Patients with schizophrenia were found to have significantly lower responses to stimulation at all concentrations of anti-CD3 (0.01 μg/ml anti-CD3 p=0.0007, 0.1 μg/ml anti-CD3 p=0.001, 1 μg/ml anti-CD3 p=0.001) when analysed using a non-parametric Mann-Whitney U test. These samples were taken from a combination of chronic patients treated with antipsychotic medication, minimally treated patients, individuals non-compliant with drug therapy and from drug naïve first onset psychosis patients. In order to exclude the possibility that lower proliferative responses were a drug effect, changes in drug naïve and minimally treated individuals were examined separately. ³H-thymidine incorporation was measured in 11 untreated/minimally treated patients and 12 matched controls, stimulated by the same method, also showing a lower proliferative response to stimulation with anti-CD3 at concentrations of 0.1 μg/ml (p=0.034) and 1 μg/ml (p=0.034).

### Lower Proliferative Responses in Schizophrenia T-Cells are not a Result of Lower CD3 Expression

CD3 expression was measured in T-cells from patients and controls before and after stimulation using an antibody against CD3ε, conjugated to Cy5. There was no significant difference in the expression of CD3 on T-cells between patients and controls both in unstimulated cells and in those treated with anti-CD3, indicating that the lower proliferative responses of patients were not a result of lower CD3 expression.

Analysis of Differentially Expressed Genes in Freshly Isolated T-Cells from Schizophrenia Patients and Matched Controls

T-cell proliferative responses to stimulation with anti-CD3 can be influenced by many factors. The processes involved in T-cell activation include cell signalling, gene transcription, protein synthesis and trafficking, entry into cell cycle and cytokine secretion. Dysfunction in any of these may result in the observed lower proliferative responses of patients. Preliminary studies were initially conducted to investigate upstream signalling, 5 minutes after T-cell stimulation with anti-CD3. This was carried out by Western blot analysis on T-cell lysates using an antibody raised against global phospho-tyrosine. No differences were evident in the patterns of tyrosine phosphorylation between patients and controls, suggesting that deficits responsible for the lower proliferative responses lie in downstream events following T-cell stimulation. Cytokine production, in particular IL-2 which drives T-cell proliferation, was also investigated following T-cell stimulation and did not show significant differences between patients and controls (manuscript in preparation). CodeLink™ Human Whole Genome microarrays were used to profile gene expression in peripheral blood T-cells from patients and controls, in order to identify altered gene expression that could underlie lower proliferative responses in patients.

Following hybridisation and scanning, data sets were filtered to include only those genes which flagged ‘good’, or present, on 100% microarray slides. Paired t-tests were then used to identify significantly differentially expressed genes, resulting in 399 probes significant at q<0.05 after multiple testing correction. 320 (80%) probes were decreased in schizophrenia and 79 were increased.

### Functional Profiling of Significantly Altered Transcripts

OntoExpress was used to assign functional categories to the significantly altered genes and to identify pathways that were over-represented in the list of significant genes. This analysis revealed five significant categories pertaining to cell cycle, including cell cycle (p=0.0005), cell cycle arrest (p=0.0007), negative regulation of cell cycle (p=0.001), mitosis (p=0.005) and regulation of cell cycle (p=0.039) (Table 2).

Categories pertaining to cell cycle were significantly altered in freshly isolated, unstimulated T-cells. Dysregulation of a number of transcripts involved in governing the progression through cell cycle was observed; these included STAT1, RBL1 (p107), RBL2 (p130), Cul2 and GADD45A, found to be up-regulated in the present study, which is a cell cycle checkpoint gene, responsible for halting G2/M progression following UV damage (6).

Four categories relating to intracellular signalling were identified by functional profiling, which may interfere with proliferative responses to anti-CD3 by affecting upstream pathways. Intracellular signalling is one of the most fundamental functions of a cell, crucial for mosT-cellular processes including energy utilisation, responses to growth factors and neurotransmitter signalling. The four pathways pertaining to cell signalling were signal transduction (p=0.0009), cell-cell signalling (p=0.012), protein amino acid phosphorylation (p=0.015), intracellular signalling cascade (p=0.050). See Table 3.

Other significantly altered signalling transcripts were CDC2L5 and NLK (down-regulated 1.42-fold and 1.54-fold, respectively). Both are also associated with cell cycle.

Transcripts for MAP2K1 were down-regulated 1.37-fold in schizophrenia patients. In T-cell stimulation, activation via the T-cell receptor (TCR) results ultimately in gene transcription and proliferation, involving cross talk of a number of signalling pathways including MAPK, activating IL-2 gene transcription and members of the protein kinase C family (PKC). PKC theta and PKC epsilon, both members of the novel PKC family, requiring DAG but not calcium for activation, were up-regulated 1.24-fold and down-regulated 1.47-fold, respectively, in schizophrenia.

Other categories that were revealed as significantly altered by OntoExpress included response to oxidative stress (p=0.0003), electron transport (p=0.001) and metabolism (p=0.013). See Table 4. Members of these functional categories showed a bias towards down-regulation of expression in schizophrenia patient T-cells. Overall, 80% of transcripts were down-regulated, mirroring alterations in protein expression from the prefrontal cortex post mortem brain study, which showed major downregulation of proteins associated with mitochondria and oxidative stress (9).

There was increased expression of the antioxidants glutathione peroxidase 7, thioredoxin 2 and ferredoxin reductase. Down-regulation of thioredoxin reductase 2 was observed. Down-regulation of methionine sulfoxide reductase was also identified. This is a protein repair enzyme, responsible for the reduction of oxidised methionine side chain in proteins, which can impair normal function. Alterations in the expressions of antioxidants and radical scavengers can be indicative of oxidative stress.

### Chromosomal Location of Significantly Altered Transcripts

In order to further understand the differences in gene expression between schizophrenia patients and controls, a heat map was generated, visualising the chromosomal locations of differentially expressed genes. Clusters of genes significantly altered between patients and controls from freshly isolated T-cells were identified at chromosomal regions 1p36, 1q42, 4q12, 6p22, 9q22 and 10q26. 1p36, 1q42 and 6p22 are strong susceptibility loci for schizophrenia (OMIM).

### Example 7

In this Example, SELDI proteomic profiling of T-cells was performed, on 15 schizophrenia patients and 15 matched healthy controls. Lysates were made from unstimulated T-cells and anti-CD3-stimulated T-cells were cultured for 48 hours, to compare the respective responses.

Lysates were profiled using CM10 chips with a weak cationic exchange surface. Very stringent binding conditions (9 M urea, 50 mM Hepes, 2% chap pH7) were used. This ensures binding only of strongly cationic proteins at pH7 (less stringent at lower pH). This investigates only a small part of proteome but enhances the chances of protein identification.

PCA analysis showed differentially expressed peaks contributing to separation of patient groups from control groups. These included, with possible products shown in brackets: 3242 Da (=Histone 1.4), 3450 Da (=alpha defensin 1), 3374 Da (=alpha defensin 1), 10918 Da, 13791 Da, and 6700 Da.

Peak identification was conducted as follows:

<4 KDa direct on chip sequencing SELDI TOF TOF (Ciphergen Calif.)

>4 KDa sequencing with LC MS MS

Large proteins have to be proteolytically processed for LC MS MS identification. As this results in a mixture of peptides for each protein, protein mix needs to be very simple in order to relate peptides back to original protein.

Defensins are known to contain 3 disulphide bonds. This is confirmed in that, using 10 mM DTT, nearly complete reduction and a shift of 6 KDa can be seen.

TABLE 2
CELL CYCLE

| GO ID/Gene Symbol | Function Name/Gene Title | Fold Change | Corrected P-Value/Qvalue |
|---|---|---|---|
| GO:0007049 | cell cycle | | 4.88E−04 |
| CNAP1 | chromosome condensation-related SMC-associated protein 1 | 1.37 | 0.04325882 |
| RBL2 | retinoblastoma-like 2 (p130) | 1.30 | 0.047737 |
| CUL2 | cullin 2 | 1.22 | 0.04954768 |
| FHIT | fragile histidine triad gene | −1.40 | 0.0195903 |
| RAD21 | RAD21 homolog (S. pombe) | −1.40 | 0.02232591 |
| RFP2 | ret finger protein 2 | −1.27 | 0.03579506 |
| HDAC4 | histone deacetylase 4 | −1.29 | 0.04053407 |
| STAG1 | stromal antigen 1 | −1.38 | 0.04954768 |
| GO:0007050 | cell cycle arrest | | 7.35E−04 |
| GADD45A | growth arrest and DNA-damage-inducible, alpha | 1.24 | 0.03725377 |
| CUL2 | cullin 2 | 1.22 | 0.04954768 |
| DST | dystonin | −1.43 | 0.02410388 |
| NOTCH2 | Notch homolog 2 (Drosophila) | −1.35 | 0.03160771 |
| GO:0045786 | negative regulation of cell cycle | | 0.001112006 |
| RBL2 | retinoblastoma-like 2 (p130) | 1.30 | 0.047737 |
| RBL1 | retinoblastoma-like 1 (p107) | −1.51 | 0.01947636 |
| FHIT | fragile histidine triad gene | −1.40 | 0.0195903 |
| RFP2 | ret linger protein 2 | −1.27 | 0.03579506 |
| GO:0007067 | mitosis | | 0.005256724 |
| CNAP1 | chromosome condensation-related SMC-associated protein 1 | 1.37 | 0.04325882 |
| RAD21 | RAD21 homolog (S. pombe) | −1.40 | 0.02232591 |
| STAG1 | stromal antigen 1 | −1.38 | 0.04954768 |
| GO:0000074 | regulation of cell cycle | | 0.038588133 |
| STAT1 | signal transducer and activator of transcription 1, 91 kDa | 1.33 | 0.02410388 |
| MPHOSPH9 | M-phase phosphoprotein 9 | 1.26 | 0.04843386 |
| PGF | placental growth factor; vascular endothelial growth factor-related protein | −1.28 | 0.02723825 |

TABLE 3
INTRACELLULAR SIGNALLING

| GO ID/Gene Symbol | Function Name/Gene Title | Fold Change | Corrected P-Value/Qvalue |
|---|---|---|---|
| GO:0007165 | signal transduction | 1503 | 9.13E−04 |
| OR7E35P | olfactory receptor, family 7 subfamily E, member 35 pseudogene | 1.28 | 0.02272425 |
| BRE | brain and reproductive organ-expressed (TNFRSF1A modulator) | 1.22 | 0.03893557 |
| FEZ2 | fasciculation and elongation protein zeta 2 (zygin II) | 1.35 | 0.03933328 |
| MGST2 | microsomal glutathione S transferase 2 | 1.36 | 0.04987676 |
| SAMHD1 | SAM domain and HD domain 1 | −1.36 | 0.01947636 |
| DTNA | dystrobrevin, alpha | −1.42 | 0.02225044 |
| NPAS2 | neuronal PAS domain protein 2 | −1.28 | 0.02232591 |
| MRGPRX3 | MAS-related GPR, member X3 | −1.33 | 0.02272425 |
| GNAQ | Guanine nucleotide binding protein (G protein), q polypeptide | −1.41 | 0.02353237 |
| ALCAM | activated leukocyte cell adhesion molecule | −1.52 | 0.02465963 |
| PGF | placental growth factor, vascular endothelial growth factor-related | −1.28 | 0.02723825 |
| | protein | | |
| ANK1 | ankyrin 1, erythrocytic///ankyrin 1, erythrocytic | −1.26 | 0.03052377 |
| CCL5 | chemokine (C-C motif) ligand 5 | −1.38 | 0.03579506 |
| ITPR1 | inositol 1,4,5-triphosphate receptor, type 1 | −1.27 | 0.03893557 |
| ANK2 | ankyrin 2, neuronal | −3.03 | 0.04325882 |
| GNB1 | guanine nucleotide binding protein (G protein), beta polypeptide 1 | −1.26 | 0.04325882 |
| CARD4 | caspase recruitment domain family, member 4 | −1.35 | 0.04551593 |
| MAP2K1 | mitogen-activated protein kinase kinase 1 | −1.37 | 0.004880499 |
| GO:0007267 | cell-cell signaling | 331 | 0.011641162 |
| MGST2 | microsomal glutathione S-transferase 2 | 1.36 | 0.04987676 |
| DLGAP1 | discs, large (Drosophila) homolog-associated protein 1 | −1.42 | 0.01947636 |
| DHH | desert hedgehog homolog (Drosophila) | −1.43 | 0.02030222 |
| PGF | placental growth factor, vascular endothelial growth factor-related | −1.28 | 0.02723825 |
| | protein | | |
| CCL5 | chemokine (C-C motif) ligand 5 | −1.38 | 0.03579506 |
| GO:0006468 | protein amino acid phosphorylation | 628 | 0.015267307 |
| PRKCQ | protein kinase C, theta | 1.24 | 0.03933328 |
| GSK3B | glycogen synthase kinase 3 beta | 1.22 | 0.04116277 |
| PTK2 | PTK2 protein tyrosine kinase 2 | −1.46 | 0.01947636 |
| NLK | nemo like kinase | −1.54 | 0.02232591 |
| PRKCE | protein kinase C, epsilon | −1.47 | 0.02710819 |
| CDC2L5 | cell division cycle 2-like 5 (cholinesterase-related cell division | −1.42 | 0.03124629 |
| | controller) | | |
| MAP2K1 | mitogen-activated protein kinase kinase 1 | −1.37 | 0.04880499 |
| GO:0007242 | intracellular signaling cascade | 451 | 0.049500096 |
| STAT1 | signal transducer and activator of transcription 1, 91 kDa | 1.33 | 0.02410388 |
| PRKCQ | protein kinase C, theta | 1.24 | 0.03933328 |
| PRKCE | protein kinase C, epsilon | −1.47 | 0.02710819 |
| USH1C | Usher syndrome 1C (autosomal recessive, severe) | −1.20 | 0.04752731 |

TABLE 4
OXIDATIVE STRESS

| GO ID/Gene Symbol | Function Name/Gene Title | Fold Change | Corrected P-Value/Qvalue |
|---|---|---|---|
| GO:0006979 | response to oxidative stress | | 2.73E−04 |
| GPX7 | glutathione peroxidase 7 | 1.31 | 0.03486197 |
| MSRA | methionine sulfoxide reductase A | −1.31 | 0.02934341 |
| CCL5 | chemokine (C-C motif) ligand 5 | −1.38 | 0.03579508 |
| C10orf120 | chromosome 10 open reading frame 120 | −1.30 | 0.03803029 |
| GO:0006118 | electron transport | | 9.89E−04 |
| TXN2 | thioredoxin 2 | 1.29 | 0.02465963 |
| FDXR | ferredoxin reductase | 1.26 | 0.0319924 |
| COX5A | cytochrome c oxidase subunit Va | 1.23 | 0.03933328 |
| ACADVL | acyl-Coenzyme A dehydrogenase, very long chain | 1.20 | 0.04953246 |
| ACAD9 | acyl-Coenzyme A dehydrogenase family, member 9 | −1.41 | 0.01947636 |
| CYB5 | cytochrome b-5 | −1.22 | 0.03586909 |
| TXNRD2 | thioredoxin reductase 2 | −1.39 | 0.03799082 |
| ERO1LB | ERO1-like beta (S. cerevisiae) | −1.35 | 0.04342341 |
| GO:0008152 | metabolism | | 0.013130081 |
| QDPR | quinoid dihydropteridine reductase | 1.25 | 0.03339055 |
| HSDL2 | hydroxysteroid dehydrogenase like 2 | 1.25 | 0.03636975 |
| CBR1 | carbonyl reductase 1 | 1.28 | 0.047737 |
| CDYL | chromodomain protein, Y-like | 1.24 | 0.04979975 |
| ATP8A1 | ATPase, aminophospholipid transporter (APLT), Class I, type 8A, member 1 | −1.37 | 0.02232591 |
| ANK2 | ankyrin 2, neuronal | −3.03 | 0.04325882 |

### REFERENCES

1. Gentleman et al, Genome Biol 2004; 5(10):R80.
2. Smyth et al, Methods 2003; 31(4):265-73.
3. Smyth et al, Stat Appl Genet Mol Biol 2004; 3(1):Article3.
4. Storey et al, Proc Natl Acad Sci USA 2003; 100(16):9440-5.
5. Draghici et al, Nucleic Acids Res 2003; 31(13):3775-81.
6. Gupta et al, Oncogene 2005; 24(48):7170-9.
7. Marques et al, Biochem Biophys Res Commun 2000; 279(3):832-7.
8. Brott et al, Proc Natl Acad Sci USA 1998; 95(3):963-8.
9. Prabakaran et al, Mol Psychiatry 2004; 9(7):684-97, 643.
10. Marchbanks et al, Schizophr Res 2003; 65(1):33-8.
11. Zhang et al, J Psychiatr Res 2002; 36(5):331-6.
12. Muller et al, Eur Arch Psychiatry Clin Neurosci 1999; 249 Suppl 4:62-8.
13. Reif et al, Mol Psychiatry 2006; 11(5):514-22.
14. Regis et al, Trends Immunol 2006; 27(2):96-101.
15. Xi et al, J Natl Cancer Inst 2006; 98(3):181-9.
16. Litovchick et al, Mol Cell Biol 2004; 24(20):8970-80.
17. Smith et al, Cell Growth Differ 1998; 9(4):297-303.
18. Garcia et al, Science 2000; 288(5474):2226-30.
19. Dudley et al, Science 2003; 301(5631):379-83.
20. Rutter et al, Science 2001; 293(5529):510-4.
21. Grima et al, Schizophr Res 2003; 62(3):213-24.
22. Koerkamp et al, Mol Biol Cell 2002; 13(8):2783-94.
23. Carlsten et al, J Comp Neurol 2001; 432(2):155-68.
24. Weickert et al, Arch Gen Psychiatry 2004; 61(6):544-55.
25. Talbot et al, J Clin Invest 2004; 113(9):1353-63.
26. Benitez-King et al, Curr Drug Targets CNS Neurol Disord 2004; 3(6):515-33.
27. Garey et al, J Neurol Neurosurg Psychiatry 1998; 65(4):446-53.
28. Glantz et al, Arch Gen Psychiatry 2000; 57(1):65-73.
29. van Woerkom, Med Hypotheses 1990; 31(1):7-15.
30. Levite et al, Eur J Immunol 2001; 31(12):3504-12.

1-63. (canceled)

64. A method of diagnosing or monitoring a psychotic disorder in a subject, or of monitoring efficacy of a therapeutic substance in a subject having, suspected of having, or of being predisposed to, a psychotic disorder, wherein said method comprises:
a. providing a test T-cell sample from the subject;
b. providing a stimulus to the test T-cell sample; and
c. assessing a response to the stimulation.

65. The method according to claim 64, further comprising comparing the response with a response to stimulation in a control sample, wherein the control sample comprises a psychotic disorder control T-cell sample or a normal T-cell control sample.

66. The method according to claim 64, wherein the assessing comprises analysing T-cell proliferation, and wherein a lower proliferation in the test T-cell sample compared to a normal control T-cell sample is indicative of the presence of a psychotic disorder or a predisposition thereto.

67. The method according to claim 64, wherein the assessing comprises analysing mRNA activity, preferably by reverse transcription and polymerase chain reactions (RT-PCR) or by quantitative reverse transcription and polymerase chain reactions (QRT-PCR); or analysing protein and/or enzyme activity; or analysing by a method selected from iTRAQ or mass spectrometry, NMR, SELDI (-TOF) and/or MALDI (-TOF), 1-D gel-based analysis, 2-D gel-based analysis, LC-MS-based technique label-free quantitative LC-MS/MS, an immunological technique and NMR.

68. The method according to claim 64, used for monitoring efficacy of a therapeutic substance in a subject having, suspected of having, or of being predisposed to, a psychotic disorder.

69. A method of identifying a biomarker of a psychotic disorder, comprising:
a. providing a test T-cell sample from a subject having a psychotic disorder;
b. providing a stimulus to the test T-cell sample;
c. assessing a response to the stimulus;
d. comparing the response with a response to stimulus in a control T-cell sample; and
e. detecting any difference in the responses, thereby identifying a biomarker.

70. The method according to claim 69, wherein the test T-cell sample is from a subject having a first psychotic disorder and the control T-cell sample is from a subject having a second psychotic disorder, or from a normal subject.

71. The method according to claim 69, wherein the assessing comprises analysing gene expression; or analysing by a method selected from iTRAQ or mass spectrometry, NMR, SELDI (-TOF) and/or MALDI (-TOF), 1-D gel-based analysis, 2-D gel-based analysis, LC-MS-based technique label-free quantitative LC-MS/MS, an immunological technique and NMR.

72. A method of testing for a potential agent for therapy of a psychotic disorder, which comprises:
a. providing a test T-cell sample from a subject having a psychotic disorder;
b. contacting the test T-cell sample with a candidate agent;
c. providing a stimulus to the test T-cell sample; and
d. assessing a response to the stimulation.

73. The method according to claim 72, further comprising comparing the response with a response to stimulation in a control sample, and identifying the candidate as a potential therapeutic agent if the response in the test sample is modulated, wherein the control sample comprises a psychotic disorder sample or a normal control sample.

74. The method according to claim 64, wherein the stimulus is a stimulus for T-cell proliferation.

75. The method according to claim 64, wherein the stimulus is an anti-CD3 antibody.

76. The method according to claim 72, wherein the response comprises modulation of gene expression or T-cell proliferation, and wherein the T-cell proliferation is assessed by ³[H]-thymidine incorporation into progeny cell DNA.

77. The method according to claim 76, wherein the assessing comprises analysing the level of one or more proteins, or wherein the assessing comprises RT-PCR or QT-PCR.

78. The method according to claim 64, wherein the psychotic disorder is bipolar disorder or schizophrenia.

79. The method, according to claim 69, wherein the stimulus is a stimulus for T-cell proliferation or is an anti-cD3 antibody.

80. The method, according to claim 72, wherein the stimulus is a stimulus for T-cell proliferation or is an anti-cD3 antibody.

81. The method, according to claim 69, wherein the psychotic disorder is bipolar disorder or schizophrenia.

82. The method, according to claim 72, wherein the psychotic disorder is bipolar disorder or schizophrenia.
